(19) United States
(12) Patent Application Publication
(10) Pub. No.: US 2008/0208317 A1
(43) Pub. Date: Aug. 28, 2008

(54) Anchoring Device for Stent

(75) Inventors: Yang-Soo Jang, Seoul (KR); Yong-Sun Ryu, Gyoungki-do (KR); Jae-Mo Ahn, Gyoungki-do (KR)

Correspondence Address: MERCHANT & GOULD PC, P.O. BOX 2903, MINNEAPOLIS, MN, 55402-0903, US

(73) Assignee: Humed Co., Ltd, Yongsan-ku (KR)

(21) Appl. No.: 11/997,573

(22) PCT Filed: May 29, 2006
(86) PCT No.: PCT/KR2006/002042

§ 371 Date: Feb. 1, 2008

(30) Foreign Application Priority Data
Aug. 2, 2005 (KR) 10-2005-0070696

(51) Int. Cl. A61F 2/82

(52) U.S. Cl. 623/1.15

(57) ABSTRACT

Disclosed is a stent supporting device. The support device anchors a stent in a manner such that an anchoring stent is inserted into a branch blood vessel adjacent to an artery in which the stent is disposed, and the anchoring stent is connected to the stent. Accordingly, the support device is safe because it does not use subsidiary members such as screws, which can injure a blood vessel. Further, since the support device allows the stent to have a length corresponding to the size of a treatment site, that is, the stent does not need to be longer than the size of the treatment site, it is easy to install the stent.

### TECHNICAL FIELD

The present invention relates to a stent supporting device, and more particularly to a stent supporting device, capable of securely supporting a stent lest the stent move.

### BACKGROUND ART

Generally, a stent is a medical instrument used to treat aneurysms, thrombosis, embolisms, and so on, and is a tubular structure disposed in a lumen of a vessel. The stent is made of metal having super-elastic and springy characteristics, such as nitinol.

The stent is inserted into a lumen of a blood vessel using a catheter in a not-expanded state, and is then expanded at a target location, either using a balloon or automatically.

A balloon-expandable stent is used together with a catheter equipped with a balloon. The stent is inserted into a blood vessel along with a catheter in a state in which it is placed around the outer circumferential surface of a balloon, is moved to a target position, and then expands as the balloon expands, thereby outwardly pressing the wall of a blood vessel. After the stent is expanded, the balloon is contracted and then removed from the blood vessel along with the catheter. After the balloon is removed, the stent is permanently disposed at the target location, improving blood flow by expanding the blood vessel.

An automatically expandable stent has a relatively large diameter in a contracted state. Accordingly, this kind of stent is used to treat blood vessels having relatively large diameters, such as a carotid artery or popliteal artery. This stent is transferred to a target position in a blood vessel in a state in which it is loaded in a catheter, and is pushed out from the inside of the catheter using an additional instrument inserted into the catheter, so that the stent is disposed at the target location. This stent has strong elastic resilience, so that it can endure high pressure and not be easily deformed.

There are many conventional stents having a variety of shapes. For example, a conventional stent has a tubular shape, in which a plurality of hoops is sequentially coupled along the central axial direction of the stent.

Each of the hoops comprises a plurality of struts, which are made of a springy metal or a highly elastic metal and are arranged in a zigzag manner along a circumferential direction. Each of the hoops also comprises a plurality of loops, each connecting the facing ends of neighboring struts.

Each of the hoops has a ring shape, which is formed by connecting the plurality of struts to each other in a ring arrangement, so that each of the hoops has a radial force by which each hoop can automatically expand, and has a structure in which it can be easily contracted inward.

If an external force is applied to the outer surface of the stent, the struts arranged in a zigzag manner are plastically deformed to come into closer contact with each other, so that the stent is contracted to have a smaller diameter. As a result, the stent has a size that can be easily implanted into a blood vessel by being loaded in a catheter.

The stent, transferred to a target location and then unloaded from a catheter, is expanded by radial force due to the elasticity of the struts of respective hoops, so that the stent increases in diameter, thereby outwardly pressing the wall of a blood vessel at the target location and becoming fixedly disposed in the target location in the blood vessel.

In order to treat a localized aneurysm, a pathological condition defined as blood-filled dilation of a blood vessel caused by a disease or weakening of the vessel's wall, a stent graft, made of a piece of thin tubular-shaped material called a graft, is attached to a stent. The stent graft is inserted into a blood vessel using a catheter or a delivery device, and is then fixed at a target site to be treated in the blood vessel. After that, the stent graft expands in the radial direction and becomes anchored to the wall of the blood vessel, so that it can remain in the target position in the blood vessel.

### DISCLOSURE OF INVENTION

### Technical Problem

A conventional stent graft is longer than the size of a target site, an aneurysm, in an artery because it should be disposed at the target site for treatment and should also be disposed at an area peripheral to the target site for fixation of the stent.

Installation of a stent graft longer than the size of a target site into an artery is very hard and troublesome. Since an artery is the blood vessel having the largest diameter in a human body, the stent graft, once anchored in an artery, can slip away and migrate from its original installation place as time passes, so that complications arise.

In order to solve the above problem, stent grafts have been anchored to the wall of a blood vessel using a plurality of screws. However, in the case of using such anchoring method, the site at which the stent graft is anchored bleeds because the wall of the blood vessel is injured by the screws, or complications arise at the anchoring site due to the bleeding.

Accordingly, the present invention has been devised in consideration of the aforementioned problems and situations, and it is an object of the present invention to provide a stent supporting device capable of anchoring a stent so that it does not slip away from its initial installation site, without injuring the wall of a blood vessel.

### Technical Solution

In order to achieve the above objects and advantageous effects, according to one aspect of the present invention, a stent supporting device implanted into an aorta comprises an anchoring stent inserted into and fixed to a branch blood vessel adjacent to the aorta into which the stent is implanted; and a connection member for connecting the anchoring stent and the stent.

The connection member may connect a peak of a hoop of the stent and a peak of a hoop of the anchoring stent among the most adjacent ends of the stent and the anchoring stent.

The connection member may be a wire, and the wire ties the stent and the anchoring stent, producing a predetermined space between the stent and the anchoring stent, so that the stent and the anchoring stent are connected to rotate freely around each other.

The connection member may be a ring, and the ring connects the stent and the anchoring stent, producing a predetermined space between the stent and the anchoring stent, so that the stent and the anchoring stent are connected to rotate freely around each other.

### ADVANTAGEOUS EFFECTS

The stent supporting device according to the present invention is configured in a manner such that the anchoring stent is inserted into a branch blood vessel adjacent to an artery in which a stent is disposed, and the anchoring stent is connected to the stent so that the anchoring stent supports the stent. Accordingly, the stent does not become displaced from its initial installation position, such displacement occurring when the stent slips. Further, the stent supporting device according to the present invention is safe because it does not use screws, which can injure a blood vessel.

In the case of using the stent supporting device according to the present invention, the stent does not have to have extra length for anchoring. That is, since the stent may have a length corresponding to the size of a target site to be treated, the stent can be easily installed.

### BRIEF DESCRIPTION OF THE DRAWINGS

FIG. 1 is a schematic view illustrating a stent supporting device according to the present invention in which the stent supporting device is installed in an abdominal aorta;

FIG. 2 is a schematic view illustrating a stent supporting device according to the present invention in which the stent supporting device is installed in an aortic arch of a heart;

FIG. 3 is an enlarged view of part “A” in FIG. 1; and

FIG. 4 is a view illustrating the connection member shown in FIG. 3.

### MODE FOR THE INVENTION

Hereinafter, a stent supporting device according to the present invention will be described with reference to the accompanying drawings.

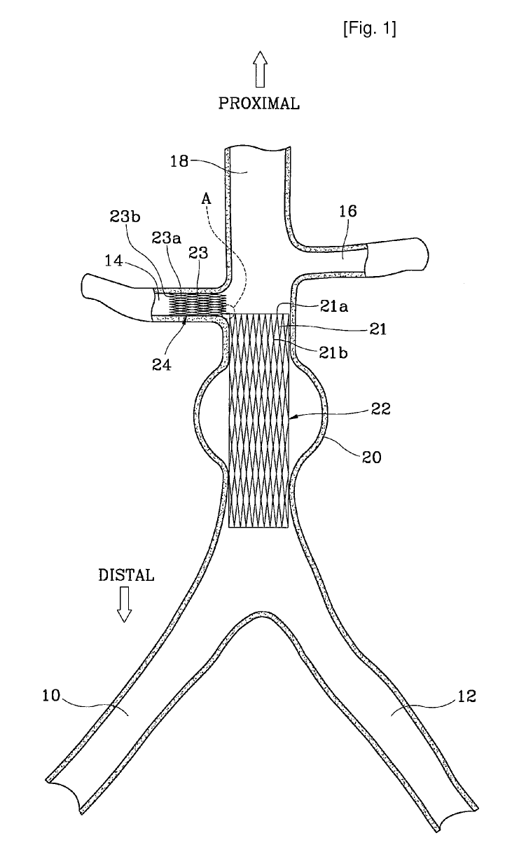

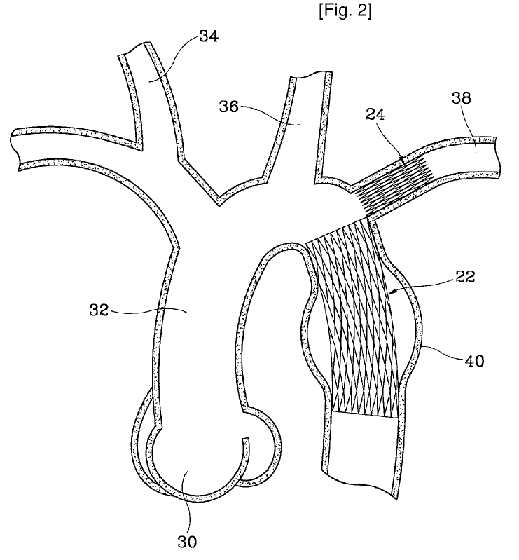

FIG. 1 illustrates a stent supporting device according to the present invention, installed in an abdominal aorta, and FIG. 2 illustrates a stent supporting device according to the present invention, installed in an aortic arch of a heart.

Referring to FIG. 1, reference numeral 10 denotes a right iliac artery, reference numeral 12 denotes a left iliac artery, reference numeral 14 denotes a right renal artery, reference numeral 16 denotes a left renal artery, reference numeral 18 denotes an abdominal aorta, and reference numeral 20 denotes an abdominal aortic aneurysm.

In FIG. 1, when the abdominal aorta 18, extending between a proximal side and a distal side, is a main blood vessel, the right renal artery 14 and the left renal artery 16, branching left and right from the abdominal aorta 18, are branch blood vessels.

Referring to FIG. 2, reference numeral 30 denotes a heart, reference numeral 32 denotes an aorta, reference numeral 34 denotes a right carotid artery, reference numeral 36 denotes a left carotid artery, reference numeral 38 denotes a left subclavian artery, and reference numeral 40 denotes an aneurysm of the aorta.

In FIG. 2, when the aorta 32 is a main blood vessel, the left carotid artery 36 and the left subclavian artery 38 branching from the aorta 32 are branch blood vessels.

As shown in FIG. 1 and FIG. 2, the stent supporting device according to the present invention is installed to branch blood vessels in a heart or a kidney, etc.

The stent supporting device according to the present invention comprises a stent 22 inserted into the artery and fixed at a site to be treated, such as the abdominal aortic aneurysm 20, an anchoring stent 24 inserted into and fixed to a branch blood vessel such as the right renal artery 14, adjacent to the stent installation site, and a connection member for connecting the stent 22 to the anchoring stent 24.

The stent 22 has a tubular shape, which is configured by connecting a plurality of hoops 21 having a ring shape in a longitudinal direction. Each hoop 21 comprises a plurality of struts arranged in a zigzag manner along a circumferential direction, forming a plurality of peaks 21a and valleys 21b. Accordingly, the stent 22 can expand and contract because it has self-elasticity.

The stent 22 can have a graft thereon according to the treatment site or treatment purpose. For example, in order to treat an aneurysm of the aorta, which is the ballooning of the inner wall of an aorta, occurring when the wall of an aorta is injured or diseased, a stent to which a graft is attached is used. The stent with a graft is called a stent graft.

The anchoring stent 24 has the same structure as the stent 22 but has a size adaptable to a branch blood vessel. In more detail, the anchoring stent 24 has a tubular shape in which a plurality of hoops 23, each having a ring shape, are connected in a longitudinal direction. Each hoop 23 comprises a plurality of struts arranged in a zigzag manner along a circumferential direction to form a plurality of peaks 23a and valleys 23b. Accordingly, the anchoring stent 24 can expand and contract because it has self-elasticity.

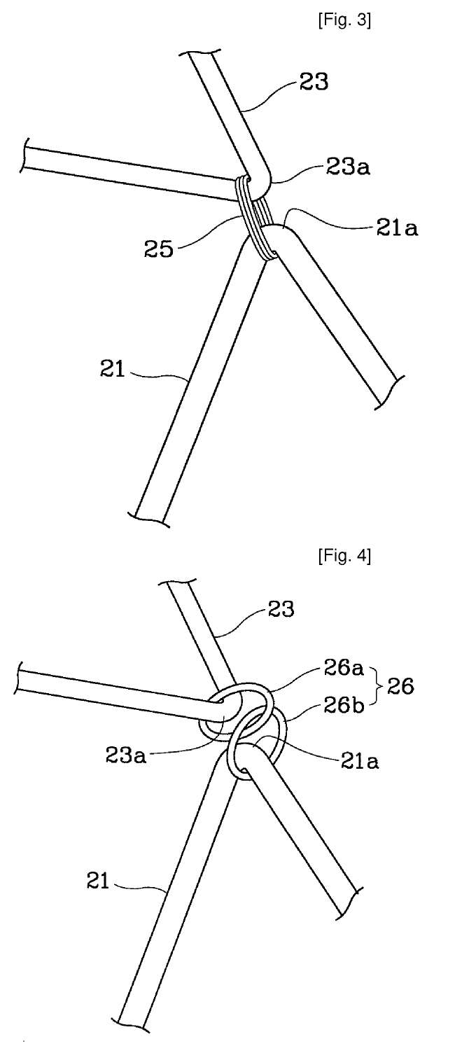

The connection member serves to connect the most adjacent ends of the stent 22 and the anchoring stent 24. Referring to FIG. 3, the connection member may be a wire 25 tying the peaks 21a of the hoop 21 of the stent 22 and the peaks 23 of the hoop 23 of the anchoring stent 24 among the ends of both.

The wire 25 ties the peaks 21a and the peaks 23a spacing apart so that they may not come into close contact with each other. Accordingly, the stent 22 and the anchoring stent 24 are connected to rotate freely each other and the contact points of the stent 22 and the anchoring stent 24 are not worn out by friction.

As shown in FIG. 3, the wire 25 can tie the stent 22 and the anchoring stent 24 at only one point. That is, the wire 25 can tie only one pair of peaks (the peak 21a of the hoop 21 and the peak 23a of the hoop 23). However, the stent 22 and the anchoring stent 24 can be tied at two or three points. That is, two or three pairs of peaks of adjacent hoops of the stent 22 and the anchoring stent 24 can be tied.

However, if four or more peaks are tied, the flow of blood in the main artery into which the stent 22 is implanted and in the branch blood vessel in which the anchoring stent 24 is implanted might be hindered. Thus, careful operation is required.

Alternatively, the connection member may be a ring. As shown in FIG. 4, the peak 21a of the hoop 21 of the stent 22 and the peak 23a of the hoop 23 of the anchoring stent 24, which is adjacent to the peak 21a, can be connected using a ring 26 in a manner such that the peak 21a of the hoop 21 and the peak 23a of the hoop 23 are not in close contact. That is, a predetermined space exists between the peak 21a of the hoop 21 and the peak 23a of the hoop 23.

The ring 26 connecting the peak 21a and the peak 23a may be only one. As shown in FIG. 4, the ring 26 may comprise a pair of rings 26a and 26b connected in the form of the numeral “8.” The ring 26 may comprise three or more rings connected in a chain form.

The peak of the hoop of the stent and the peak of the hoop of the anchoring stent can be connected at one point. That is, only one pair of peaks, the peak of the hoop of the stent and the peak of the hoop of the anchoring stent can be tied by the ring 26, but also two or three pairs of peaks of adjacent hoops can be tied by two or three respective rings 26. In this case, as in the case in which the stent and the anchoring stent are connected via the wire 25, tying four or more pairs of peaks of adjacent hoops must be carefully conducted so that the flow of blood is not hindered.

Hereinafter, the implantation method of the stent supporting device according to the present invention and advantageous effects thereof will be described. In the following description, it is assumed that a treatment site is an abdominal artery, referring to FIG. 1.

First, an end (the peak 21a of the hoop 21) of the stent 22 is connected to an end (the peak 23a of the hoop 23) of the adjacent anchoring stent 24 using a connection member (for example, the wire 25).

The stent 22 and the anchoring stent 24 are loaded into respective catheters (or respective delivery devices having the same function as catheters) in a contracted state, and then they are inserted together into a main artery to be treated.

The stent 22 is then delivered to a target site to be treated in an artery, and the anchoring stent 24 is inserted into a branch blood vessel that is adjacent to the target site. Next, the stent 22 and the anchoring stent 24 are expanded simultaneously or sequentially (either may be first expanded) to be fixed in the artery and the branch blood vessel, respectively.

In this instance, the anchoring stent 24 is disposed upstream relative to the stent 22 so that the anchoring stent 24 supports the stent 22 in a manner such that the anchoring stent 24 pulls the stent 22 in an upstream direction.

After the stent 22 and the anchoring stent 24 are fixed, respective catheters are pulled out simultaneously or serially from the main artery (either catheter can be pulled out first).

The stent 22 is fixed to the inner wall of the treated artery by its self-elasticity and is also supported by the anchoring stent 24 installed in the branch blood vessel. Accordingly, the stent 22 cannot be slipped or displaced.

The present invention has been explained above with reference to embodiments, but the present invention is not limited to the embodiments above. In concluding the detailed description, those skilled in the art will appreciate that many variations and modifications can be made to the preferred embodiments without substantially departing from the principles of the present invention. Therefore, it is readily understood that those variations and modifications to the preferred embodiment will be within the scope of the present invention.

1. A stent supporting device implanted into an aorta, comprising:
an anchoring stent inserted into and fixed to a branch blood vessel adjacent to the aorta into which the stent is implanted; and
a connection member for connecting the anchoring stent and the stent.

2. The stent supporting device according to claim 1, wherein the connection member connects the most adjacent ends of the stent and the anchoring stent.

3. The stent supporting device according to claim 2, wherein the connection member connects a peak of a hoop of the stent and a peak of a hoop of the anchoring stent between the ends of both.

4. The stent supporting device according to claim 1, wherein the connection member is a wire, and the wire ties the stent and the anchoring stent, producing a predetermined space between the stent and the anchoring stent, so that the stent and the anchoring stent are connected to rotate freely around each other.

5. The stent supporting device according to claim 4, wherein the connection member is a plurality of wires.

6. The stent supporting device according to claim 1, wherein the connection member is a ring, and the ring connects the stent and the anchoring stent, producing a predetermined space between the stent and the anchoring stent, so that the stent and the anchoring stent are connected to rotate freely around each other.

7. The stent supporting device according to claim 6, wherein the connection member is a pair of rings connected in the form of the numeral “8.”

8. The stent supporting device according to claim 6, wherein the connection member comprises a plurality of rings connecting the stent and the anchoring stent.

9. The stent supporting device according to claim 7, wherein the connection member comprises a plurality of ring chains connecting the stent and the anchoring stent, each chain being a pair of rings connected in the form of the numeral “8.”
